(19) United States
(12) Patent Application Publication
(10) Pub. No.: US 2009/0062277 A1
(43) Pub. Date: Mar. 5, 2009

(54) PHOSPHODIESTERASE 10 INHIBITORS

(76) Inventors: Essa Hu, Camarillo, CA (US); Roxanne Kunz, Santa Monica, CA (US); Ning Chen, Thousand Oaks, CA (US); Tom Nixey, Newbury Park, CA (US); Stephen Hitchcock, Westlake Village, CA (US)

Correspondence Address: JONES DAY, 222 EAST 41ST ST, NEW YORK, NY, 10017, US

(21) Appl. No.: 12/195,335

(22) Filed: Aug. 20, 2008

Related U.S. Application Data
(60) Provisional application No. 60/965,772, filed on Aug. 21, 2007.

(51) Int. Cl. A61K 31/5377; C07D 401/14; C07D 413/14; A61K 31/502

(52) U.S. Cl. 514/234.5; 544/235; 514/248; 544/116

(57) ABSTRACT

The present invention is directed to certain compounds useful as phosphodiesterase 10 (PDE10) inhibitors that have the formula

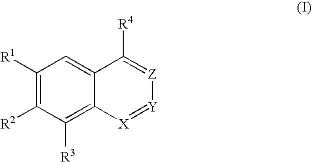

where R¹, R², R³, R⁴, X, Y and Z are as defined herein, pharmaceutical compositions containing such compounds and processes for preparing such compounds. The invention is also directed to methods of treating diseases mediated by PDE10, such as obesity, non-insulin dependent diabetes, schizophrenia, bipolar disorder, obsessive-compulsive disorder, and the like.

### CROSS REFERENCE

This application claims the benefit of U.S. Patent Application No. 60/965,772, filed Aug. 21, 2007, the disclosure of which is incorporated herein by reference in its entirety.

### FIELD OF THE INVENTION

Provided herein are certain compounds that are PDE10 inhibitors, pharmaceutical compositions containing such compounds, and processes for preparing such compounds. Provided herein also are methods of treating disorders or diseases treatable by inhibition of PDE10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activities.

Cyclic nucleotides are produced from the actions of adenylyl cyclase and guanylyl cyclase, which convert ATP to cAMP and GTP to cGMP. Extracellular signals, often through the actions of G protein-coupled receptors, regulate the activities of the cyclases. Alternatively, the amount of cAMP and cGMP may be altered by regulating the activities of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE1 is stimulated by Ca²⁺/calmodulin. PDE2 activity is stimulated by cGMP. PDE3 is inhibited by cGMP. PDE4 is cAMP specific and is specifically inhibited by rolipram. PDE5 is cGMP-specific. PDE6 is expressed in retina.

PDE10 sequences were identified by using bioinformatics and sequence information from other PDE gene families (Fujishige et al., J. Biol. Chem. 274:18438-18445, 1999; Loughney et al., Gene 234:109-117, 1999; Soderling et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because at least three alternative exons encode N termini and two exons encode C-termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunoprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, for example, in neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, provided herein is a compound of Formula (I):

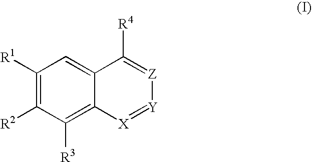

or an individual stereoisomer, a mixture of stereoisomers, or a pharmaceutically acceptable salt thereof, wherein R¹, R², R³, R⁴, X, Y and Z are defined herein.

In a second aspect, provided herein is a pharmaceutical composition comprising a pharmaceutically acceptably excipient and a compound of Formula (I), a pharmaceutically acceptable salt thereof, or a mixture of a compound of Formula (I) and a pharmaceutically acceptable salt thereof.

In a third aspect, this invention is directed to a method of treating a disorder treatable by inhibition of PDE10 in a patient which method comprises administering to the patient a pharmaceutical composition comprising a compound of Formula (I), a pharmaceutically acceptable salt thereof, or a mixture of a compound of Formula (I) and a pharmaceutically acceptable salt thereof; and a pharmaceutically acceptable excipient. Within this aspect, in one embodiment, the disease is obesity, non-insulin dependent diabetes, Huntington's disease, schizophrenia, bipolar disorder, or obsessive-compulsive disorder.

In a fourth aspect, this invention is directed the use of a compound of Formula (I), a pharmaceutically acceptable salt thereof, or a mixture of a compound of Formula (I) and a pharmaceutically acceptable salt thereof in the manufacture of a medicament for treating a disorder treatable by inhibition of PDE10 in a patient. Within this aspect, in one embodiment, the disorder is obesity, non-insulin dependent diabetes, Huntington's disease, schizophrenia, bipolar disorder, or obsessive-compulsive disorder.

It will be readily apparent to a person skilled in the art that the pharmaceutical composition could contain one or more compounds of Formula (I) (including individual stereoisomer, mixtures of stereoisomers where the compound of Formula (I) has at least one stereochemical centre), a pharmaceutically acceptable salt thereof, or mixtures thereof.

### DETAILED DESCRIPTION

### Definitions

Unless otherwise stated, the following terms used in the specification and claims are defined for the purposes of this application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“C_{α-β}alk” means an alkyl group comprising a minimum of α and a maximum of β carbon atoms in a branched, cyclical or linear relationship or any combination of the three, wherein α and β represent whole numbers. The alkyl groups described in this section may also contain one or two double or triple bonds. A designation of C₀alk indicates a direct bond. Examples of C₁₋₆alkyl include, but are not limited to the following:

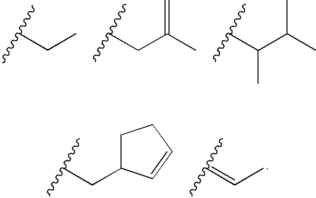

“Alkylene” means a linear saturated divalent hydrocarbon radical of one to six carbon atoms or a branched saturated divalent hydrocarbon radical of three to six carbon atoms unless otherwise stated, e.g., methylene, ethylene, propylene, 1-methylpropylene, 2-methylpropylene, butylene, pentylene, and the like.

“Alkylthio” means a —SR radical, where R is alkyl as defined above, e.g., methylthio, ethylthio, and the like.

“Alkylsulfinyl” means a —SOR radical where R is alkyl as defined above, e.g., methylsulfinyl, ethylsulfinyl, and the like.

“Alkylsulfonyl” means a —SO₂R radical, where R is alkyl as defined above, e.g., methylsulfonyl, ethylsulfonyl, and the like.

“Alkoxy” means an —OR radical, where R is alkyl as defined above, e.g., methoxy, ethoxy, propoxy, or 2-propoxy, n-, iso-, or tert-butoxy, and the like.

“Alkylamino” means an —NHR radical, where R is alkyl as defined above, e.g., methylamino, ethylamino, propylamino, or 2-propylamino, and the like.

“Alkoxycarbonyl” means a —C(O)OR radical, where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means an —OR radical, where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two —NRR′, where R is hydrogen, alkyl, or —COR^{a}, where R^{a} is alkyl, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical, where R is aminoalkyl as defined above, e.g., 2-aminoethoxy, 2-dimethylaminopropoxy, and the like.

“Aminocarbonyl” means a —CONRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., CONH₂, methylaminocarbonyl, 2-dimethylaminocarbonyl, and the like.

“Aminosulfonyl” means a —SO₂NRR′ radical, where R is independently hydrogen, alkyl, hydroxyalkyl, alkoxyalkyl, or aminoalkyl, and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —SO₂NH₂, methylaminosulfonyl, 2-dimethylaminosulfonyl, and the like.

“Acyl” means a —COR radical, where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetyl, propionyl, benzoyl, pyridinylcarbonyl, and the like. When R in a —COR radical is alkyl, the radical is also referred to herein as “alkylcarbonyl.”

“Acylamino” means an —NHCOR radical, where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetylamino, propionylamino, and the like.

“Aryl” means a monovalent monocyclic or bicyclic aromatic hydrocarbon radical of 6 to 12 ring atoms, e.g., phenyl or naphthyl.

“Aralkyl” means an -alkylene-R radical, where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantyl. Additionally, one or two ring carbon atoms may optionally be replaced with a —CO— group.

“Cycloalkylalkyl” means an -(alkylene)-R radical, where R is cycloalkyl as defined above; e.g., cyclopropylmethyl, cyclobutylmethyl, cyclopentylethyl, or cyclohexylmethyl, and the like.

“Cycloalkyloxy” means an —OR radical, where R is cycloalkyl as defined, e.g., cyclopropyloxy, cyclobutyloxy, cyclopentyloxy, cyclohexyloxy, and the like.

“Cycloalkylalkyloxy” means an —OR radical, where R is cycloalkylalkyl as defined, e.g., cyclopropylmethyloxy, cyclobutylmethyloxy, cyclopentylethyloxy, cyclohexylmethyloxy, and the like.

“Carboxy” means —COOH.

“Disubstituted amino” means an —NRR′ radical, where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined above, e.g., dimethylamino, phenylmethylamino, and the like. When R and R′ are alkyl, it is also referred to herein as dialkylamino.

Where dialkyl amines are specified, these are also intended to include cyclic alkyl amines such as

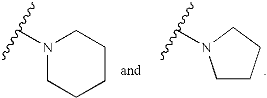

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like. When the halo atom is fluoro, it also referred to herein as fluoroalkyl.

“Haloalkoxy” means an —OR radical, where R is haloalkyl as defined above, e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that, if two hydroxy groups are present, they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means an —OR radical, where R is hydroxyalkyl as defined above.

“Heterocyclyl” means a saturated or unsaturated monovalent monocyclic group of 4 to 8 ring atoms, in which one or two ring atoms are heteroatom(s), independently selected from N, O, and S(O)ₙ, where n is an integer from 0 to 2, the remaining ring atoms are C. Additionally, the heterocyclic ring may be fused to phenyl or heteroaryl ring, provided that the entire heterocyclyl ring is not completely aromatic. Unless stated otherwise, the fused heterocyclyl ring can be attached at any ring atom. More specifically, the term “heterocyclyl” includes, but is not limited to, pyrrolidino, piperidino, homopiperidino, morpholino, piperazino, tetrahydropyranyl, thiomorpholino, and the like. When the heterocyclyl ring saturated, has five to eight ring atoms, and is not fused to phenyl or heteroaryl ring, it is also referred to herein as “5-8 membered saturated monocyclic heterocyclyl.” When the heterocyclyl ring is unsaturated, it can contain one or two ring double bonds, provided that the ring is not aromatic.

“Heterocyclylalkyl” means an (alkylene)-R radical, where R is heterocyclyl ring as defined above, e.g., tetrahydrofuranylmethyl, piperazinylmethyl, morpholinylethyl, and the like.

“Heteroaryl” means a monovalent monocyclic or bicyclic aromatic radical of 5 to 10 ring atoms, where one or more, preferably one, two, or three, ring atoms are heteroatoms independently selected from N, O, and S, and the remaining ring atoms are carbon, e.g., benzofuranyl, benzo[d]thiazolyl, isoquinolinyl, quinolinyl, thiophenyl, imidazolyl, oxazolyl, quinolinyl, furanyl, thazolyl, pyridinyl, and the like.

“Heteroaralkyl” means an (alkylene)-R radical, where R is heteroaryl as defined above.

“Monosubstituted amino” means an —NHR radical, where R is alkyl, acyl, sulfonyl, aryl, aralkyl, heteroaryl, heteroaralkyl, heterocyclyl, heterocyclylalkyl, cycloalkyl, cycloalkylalkyl, hydroxyalkyl, alkoxyalkyl, or aminoalkyl, each as defined above, e.g., methylamino, 2-phenylamino, hydroxyethylamino, and the like.

The present invention also includes prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups, however, regenerate original functional groups by routine manipulation or in vivo. Prodrugs of compounds of Formula (I) include compounds wherein a hydroxy, amino, carboxylic, or a similar group is modified. Examples of prodrugs include, but are not limited to, esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol, or any group containing a nitrogen atom, these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for instance, acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

The term “pharmaceutically acceptable salt” also refers to salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are non-toxic. Additional information on suitable pharmaceutically acceptable salts can be found in Remington's Pharmaceutical Sciences, Gennaro, A. R. (Mack Publishing Company, 18th ed., 1995), which is incorporated herein by reference.

The compounds of the present invention may have one or more asymmetric centers. Compounds of the present invention containing an asymmetrically substituted atom may be isolated in an optically active, racemic, or diastereomeric form. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof, are within the scope of this invention. It should be noted that compounds of the invention may contain groups that may exist in tautomeric forms, such as cyclic and acyclic amidine and guanidine groups, heteroatom substituted heteroaryl groups (Y′=O, S, NR), and the like, which are illustrated in the following examples:

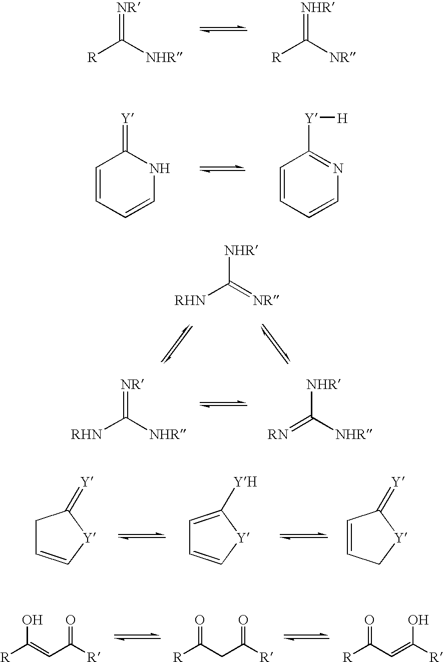

and though one form is named, described, displayed and/or claimed herein, all the tautomeric forms are intended to be inherently included in such name, description, display and/or claim.

Additionally, as used herein, the term “alkyl” includes all the possible isomeric forms of said alkyl group albeit only a few examples are set forth. Furthermore, when a cyclic group, such as aryl, heteroaryl, and heterocyclyl, is substituted, it includes all the positional isomers albeit only a few examples are set forth.

All polymorphic forms and solvates, including hydrates, of a compound of Formula (I) are also within the scope of this invention.

“Oxo” means the ═(O) group.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with an alkyl group and situations where the heterocyclyl group is not substituted with the alkyl group.

“Optionally substituted phenyl” means a phenyl ring optionally substituted with one, two, or three substituents, each independently selected from alkyl, halo, alkoxy, alkylthio, haloalkyl, haloalkoxy, amino, alkylamino, dialkylamino, hydroxy, cyano, aminocarbonyl, acylamino, alkylsulfonyl, hydroxyalkyl, alkoxycarbonyl, aminoalkyl, carboxy, cycloalkyl, cycloalkylalkyl, cycloalkoxy, cycloalkylalkyloxy, sulfinyl, and sulfonyl, each as defined herein.

“Optionally substituted heteroaryl” means a monovalent monocyclic or bicyclic aromatic radical of 5 to 10 ring atoms, where one or more, preferably one, two, or three ring atoms are heteroatoms, each independently selected from N, O, and S, and the remaining ring atoms are carbon, that is optionally substituted with one, two, or three substituents, each independently selected from alkyl, halo, alkoxy, alkylthio, haloalkyl, haloalkoxy, amino, alkylamino, dialkylamino, hydroxy, cyano, aminocarbonyl, acylamino, alkylsulfonyl, hydroxyalkyl, alkoxycarbonyl, aminoalkyl, carboxy, cycloalkyl, cycloalkylalkyl, cycloalkoxy, cycloalkylalkyloxy, sulfinyl, and sulfonyl, each as defined herein. More specifically, the term optionally substituted heteroaryl includes, but is not limited to, optionally substituted pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl, each optionally substituted as indicated above.

“Optionally substituted heterocyclyl” means a saturated or unsaturated monovalent cyclic group of 4 to 8 ring atoms in which one or two ring atoms are heteroatoms, each independently selected from N, O, and S(O)ₙ, where n is an integer from 0 to 2, and the remaining ring atoms are carbon, and/or in which one or two ring carbon atoms can optionally be replaced by a —CO— group, where the heterocyclyl is optionally substituted with one, two, or three substituents, each independently selected from alkyl, halo, alkoxy, alkylthio, haloalkyl, haloalkoxy, amino, alkylamino, dialkylamino, hydroxy, cyano, nitro, aminocarbonyl, acylamino, alkylsulfonyl, hydroxyalkyl, alkoxycarbonyl, aminoalkyl, carboxy, cycloalkyl, cycloalkylalkyl, cycloalkoxy, cycloalkylalkyloxy, sulfinyl, and sulfonyl, each as defined herein.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical, where R is alkyl, haloalkyl, aryl, aralkyl, heteroaryl, heteroaralkyl, heterocyclyl, or heterocyclylalkyl, each as defined above, e.g., methylsulfinyl, phenylsulfinyl, benzylsulfinyl, and the like.

“Sulfonyl” means a —SO₂R radical, where R is alkyl, haloalkyl, aryl, aralkyl, heteroaryl, heteroaralkyl, heterocyclyl, or heterocyclylalkyl, each as defined above, e.g., methylsulfonyl, phenylsulfonyl, benzylsulfonyl, pyridinylsulfonyl, and the like.

“Treating” or “treatment” of a disease includes:
(1) preventing the disease, i.e., causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity, and the age, weight, etc., of the mammal to be treated.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

One embodiment is a compound of Formula (I):

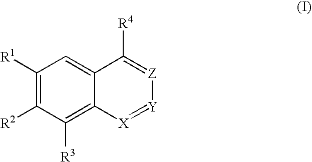

or an individual stereoisomer, a mixture of stereoisomers, or a pharmaceutically acceptable salt thereof, wherein:

one or two of X, Y and Z are —CH— and the remaining is —N—;

R¹ and R² are each independently selected from alkyl, hydroxy, or alkoxy;

R³ is hydrogen, alkyl, halo, or alkoxy;

R⁴ is a selected from formula (a) or (b):

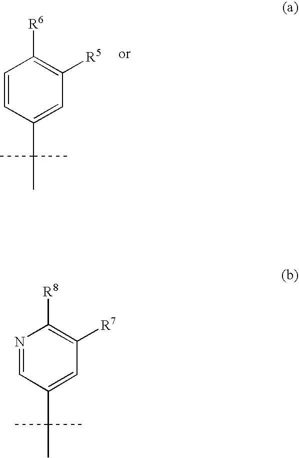

where:
R⁵ and R⁷ are independently hydrogen, alkyl, halo, or fluoroalkyl;
R⁶ and R⁸ are independently 5-8 membered monocyclic, saturated heterocyclyl substituted with one to three substituents independently selected from R^{a}, R^{b}, and R^{c} which are independently hydrogen, C₁₋₉alk, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, heterocyclylalkyl and optionally substituted heterocyclyl provided that: (i) when X and Y are N and Z is —CH═ or X and Y are —CH— and Z is —N—, then at least one of R^{a}, R^{b}, and R^{c} is not hydrogen; (ii) when X and Y are N and Z is —CH═, R⁴ is a group of formula (b) where R⁷ is hydrogen, then R⁸ is not 2-methylmorpholin-4-yl, 2,6-dimethylmorpholin-4-yl, 4-methylpiperazin-1-yl, 2,6-dimethylpiperazin-4-yl, 4-methoxypiperidin-1-yl, 4-fluoropiperidin-1-yl, 4,4-difluoropiperidin-1-yl, 4-methylaminopiperidin-1-yl, 4-hydroxy-4-phenylpiperidin-1-yl, 4-cyano-4-phenylpiperidin-1-yl, 4-hydroxypiperidin-1-yl, 1-tert-butoxycarbonylpyrrolidin-3-yl, or 3-methoxypyrrolidin-1-yl; and (iii) when X and Y are —CH═ and Z is —N—, R⁴ is a group of formula (b) where R⁷ is hydrogen, then R⁸ is not 4-methoxypiperidin-1-yl or 2,6-dimethylmorpholin-4-yl.

(1) In another embodiment, in conjunction with any above or below embodiments, X is nitrogen, and Y and Z are ═CH—.

(2) In another embodiment, in conjunction with any above or below embodiments, Y is nitrogen, and X and Z are ═CH—.

(3) In another embodiment, in conjunction with any above or below embodiments, Z is nitrogen, and X and Y are ═CH—.

(4) In another embodiment, in conjunction with any above or below embodiments, X and Y are nitrogen and Z is —CH═.

(5) In another embodiment, in conjunction with any above or below embodiments, X and Z are nitrogen and Y is —CH═.

(6) In another embodiment, in conjunction with any above or below embodiments, Y and Z are nitrogen and X is —CH═.

(A) Within the above embodiments 1-6, one group of compounds of Formula (I) is that wherein R¹ and R² are alkoxy and R³ is hydrogen. Within this group, one group of compounds is that where R¹ and R² are methoxy.

(B) Within the above embodiments 1-6, another group of compounds of Formula (I) is that wherein R¹, R², and R³ are alkoxy. Within this group, one group of compounds is that where R¹, R², and R³ are methoxy.

(i) Within the above embodiments (1)-(6), (A), (B), and embodiments contained therein, i.e., (1) (A-B), (2) (A-B), (3) (A-B), (4) (A-B), (5) (A-B), and (6) (A-B), one group of compounds of Formula (I) is that wherein R⁴ is a group of formula (a) where R⁵ and R⁶ are as defined in the Detailed Description of the Invention.

(a) Within groups (i), one group of compounds of Formula (I) is that wherein R⁵ is chloro, methyl, or difluoromethyl and R⁶ is piperidin-1-yl substituted as defined in the Detailed Description of the Invention. Within groups (i), another group of compounds of Formula (I) is that wherein R⁵ is chloro, methyl, or difluoromethyl and R⁶ is piperidin-1-yl substituted with R^{a} and R^{b} where R^{a} is hydrogen, hydroxyl, halo, or alkoxy and R^{b} is alkyl, hydroxyalkyl, alkoxyalkyl, cycloalkyl, optionally substituted phenyl or optionally substituted heteroaryl.

(ii) Within the above embodiments (1)-(6), (A), (B), and embodiments contained therein, i.e., (1) (A-B), (2) (A-B), (3) (A-B), (4) (A-B), (5) (A-B), and (6) (A-B), one group of compounds of Formula (I) is that wherein R⁴ is a group of formula (b) where R⁷ and R⁸ are as defined in the Detailed Description of the Invention.

(b) Within group (ii), one group of compounds of Formula (I) is that wherein R⁷ is hydrogen, halo, alkyl, or fluoroalkyl e.g., hydrogen, chloro, methyl, or difluoromethyl and R⁸ is piperidin-1-yl substituted as defined in the Detailed Description of the Invention. Within group (i), another group of compounds of Formula (I) is that wherein R⁷ is hydrogen, halo, alkyl, or fluoroalkyl and R⁸ is piperidin-1-yl substituted with R^{a} and R^{b} where R^{a} is hydrogen, hydroxyl, halo, or alkoxy and R^{b} is alkyl, hydroxyalkyl, alkoxyalkyl, cycloalkyl, optionally substituted phenyl, heterocycloalkyl or optionally substituted heteroaryl.

(iii) Within the above embodiments (1)-(6), (A), (B), and embodiments contained therein, i.e., (1) (A-B), (2) (A-B), (3) (A-B), (4) (A-B), (5) (A-B), and (6) (A-B), one group of compounds of Formula (I) is that wherein R⁴ is a group of formula:

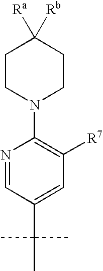

wherein R⁷ is hydrogen, halo, alkyl, or fluoroalkyl, R^{a} is hydrogen, hydroxyl, halo, amino, alkylamino, or alkoxy, and R^{b} is alkyl, hydroxyalkyl, alkoxyalkyl, aminoalkyl, cycloalkyl, optionally substituted phenyl or optionally substituted heteroaryl. Within this group, another group of compounds is that where R⁷ is hydrogen, chloro, methyl, or difluoromethyl, R^{a} is hydrogen or hydroxyl and R^{b} is alkyl, hydroxyalkyl, alkoxyalkyl, cycloalkyl, alkyl, or optionally substituted heteroaryl. Within this group, one group of compounds is that wherein R^{a} is hydrogen or hydroxyl and R^{b} is C(CH₃)(OH)CH₃, methyl, ethyl, cyclopropyl, cyclobutyl, or optionally substituted pyridin-2-yl. Within this group, one group of compounds is that wherein R^{a} is hydrogen or hydroxyl and R^{b} is —C(CH₃)(OH)CH₃, methyl, cyclopropyl, or pyridin-2-yl.

Representative compounds of Formula (I) where R¹ and R² are methoxy, R³ is hydrogen, Z is —CH—, and X and Y and R⁴ is a group of formula (b) where R⁷ and R⁸ are as provided in Table 1 below are:

TABLE 1

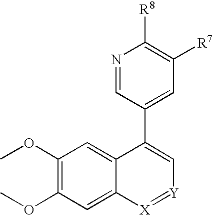

| Cpd # | X | Y | R⁷ | R⁸ |
|---|---|---|---|---|
| 1 | N | N | methyl | 4-(2-hydroxypropan-2-yl)piperidin-1-yl |
| 2 | N | N | H | 4-hydroxy-4-pyridin-2-ylpiperidin-1-yl |
| 3 | N | CH | methyl | 4-(2-hydroxypropan-2-yl)piperidin-1-yl |
| 4 | N | N | methyl | 4-cylopropyl-4-hydroxypiperidin-1-yl |
| 5 | N | N | —CHF₂ | 4-(2-hydroxypropan-2-yl)piperidin-1-yl |
| 6 | N | N | methyl | 4-hydroxy-4-pyridin-2-ylpiperidin-1-yl |
| 7 | N | N | methyl | 4-hydroxy-4-methylpiperidin-1-yl |
| 8 | N | N | chloro | 4-hydroxy-4-pyridin-2-ylpiperidin-1-yl |
| 9 | N | N | chloro | 2(R)-methoxymethylpyrrolidin-1-yl |
| 10 | N | N | H | 4-trifluoromethylpiperidin-4-yl |
| 11 | H | N | chloro | 4-(2-hydroxypropan-2-yl)piperidin-1-yl |
| 12 | H | N | methyl | 4-(2-hydroxypropan-2-yl)piperidin-1-yl |
| 13 | N | N | methyl | 4-hydroxy-4-pyridin-3-ylpiperidin-1-yl |
| 14 | N | N | H | 4-(3,5-dichloropyridin-2-yl)piperazin-1-yl |
| 15 | N | N | H | 4-(3-trifluoromethylphenyl)piperazin-1-yl |
| 16 | N | N | methyl | 4-(2,4-difluorophenyl)piperazin-1-yl |
| 17 | N | N | H | 4-(cyclopropylmethyl)piperazin-1-yl |
| 18 | N | N | H | 4-(acetyl)piperazin-1-yl |
| 19 | N | N | H | 4-(4-F-benzoyl)piperidin-1-yl |
| 20 | N | N | H | 2-dimethylaminomethylpyrrolidin-1-yl |
| 21 | N | N | methyl | 4-fluoro-4-pyridin-3-ylpiperidin-1-yl |
| 22 | N | N | H | 4-(cyclopropylcarbonyl)piperazin-1-yl |
| 23 | N | N | H | 4-(benzthiazol-3-yl)piperazin-1-yl |
| 24 | N | H | methyl | 4-(2-hydroxypropan-2-yl)piperidin-1-yl |
| 25 | N | N | H | 2-thiophen-2-ylpyrrolidin-1-yl |
| 26 | N | N | H | 4-methyl-4-phenylpiperidin-1-yl |
| 27 | N | N | H | 4-(morpholin-4-ylcarbonyl)piperidin-1-yl |
| 28 | N | N | methyl | 4-(NH₂CO)piperidin-1-yl |
| 29 | N | N | H | 4-methyl-4-(2-fluorophenyl)piperidin-1-yl |
| 30 | N | N | H | 4-ethoxycarbonylpiperidin-1-yl |
| 31 | N | CH | H | 4-hydroxy-4-phenylpiperidin-1-yl |
| 32 | N | N | H | 4-tert-butylcarbonylaminopiperidin-1-yl |

Representative compounds of Formula (I) where R³ is hydrogen, Z is —CH—, and R⁴ is a group of formula (b) where R¹, R², X, Y, R⁷ and R⁸ are as provided in Table 2 below are:

TABLE 2

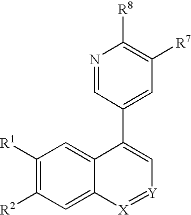

| CPD # | R¹ | R² | X | Y | R⁷ | R⁸ |
|---|---|---|---|---|---|---|
| 33 | ethoxy | methyl | N | N | methyl | 4-(2-hydroxypropan-2-yl)piperidin-1-yl |
| 34 | methyl | methoxy | N | N | methyl | 4-(2-hydroxypropan-2-yl)piperidin-1-yl |
| 35 | methoxy | methyl | N | N | H | 4-hydroxy-4-phenylpiperidin-1-yl |

### General Synthetic Schemes

Compounds of this invention can be made by the methods depicted in the reaction schemes shown below.

The starting materials and reagents used in preparing these compounds are either available from commercial suppliers, such as Aldrich Chemical Co. (Milwaukee, Wis.), Bachem (Torrance, Calif.), or Sigma (St. Louis, Mo.), or are prepared by methods known to those skilled in the art, following procedures set forth in references, such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry (John Wiley and Sons, 4th Edition), and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including, but not limited to, filtration, distillation, crystallization, chromatography, and the like. Such materials may be characterized using conventional means, including physical constants and spectral data.

Unless specified to the contrary, the reactions described herein take place at atmospheric pressure over a temperature range from about −78° C. to about 150° C., from about 0° C. to about 125° C., or at about room (or ambient) temperature, e.g., about 23° C.

Compounds of Formula (I) where R¹, R², R³, R⁴, X, Y, and Z are as defined in the Detailed Description of the Invention can be prepared as shown in Scheme 1 below.

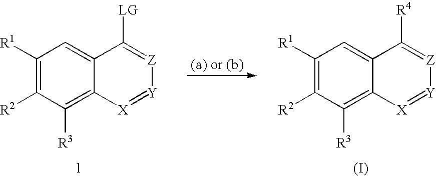

Reaction of a compound of formula 1 where LG is a suitable leaving group such as halo, with boronic acid derivative of a group of formula (a) or (b) under Suzuki type coupling conditions provide a compound of Formula (I) (see, e.g., Miyaura and Suzuki, Chem. Rev., 95:2457-2483, 1995). Such boronic acids are either commercially available (e.g., Aldrich Chemical Co. (Milwaukee, Wis.), Lancaster Synthesis (Ward Hill, Mass.), or Maybridge (Cornwall, UK)) or can readily be prepared from the corresponding bromides by methods described in the literature (see, for example, Miyaura et al., Tetrahedron Letters 20:3437-3440, 1979; Miyaura and Suzuki, Chem. Commun. 1979, 866-867).

Compounds of formula 1 where X and Y are nitrogen, Z is carbon, and R¹, R², and R³ are as defined in the Detailed Description of the Invention can be prepared as described as shown below.

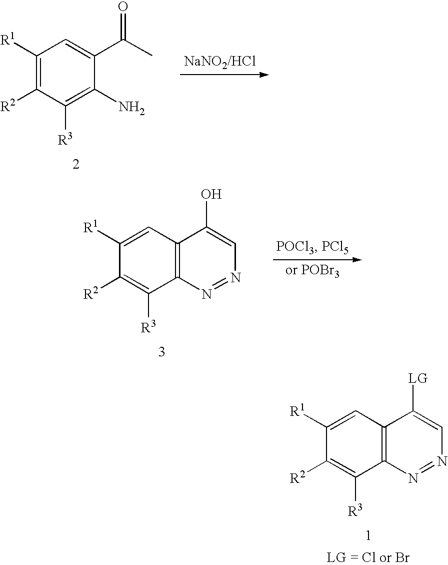

Treatment of 2-aminoacetophenone 2 with sodium nitrite in concentrated hydrochloric acid and water provides diazo compound intermediates that cyclize upon heating to provide 4-hydroxycinnolines 3. Treatment of 3 with either phosphorous oxychloride or phosphorous oxybromide provides the corresponding chloro or bromo compound of formula 1. The chloro derivative can be prepared by heating 2 in neat phosphorous oxychloride, followed by recrystallization of the product after neutralization (see, for example, Castle et al. J. Org. Chem. 17:1571, 1952). The bromo derivative can be prepared by mixing a concentrated suspension of the 4-hydroxycinnoline 3 in chloroform and phosphorous oxybromide at room temperature and then warming to reflux for 8 to 16 h. Extractive workup after neutralization and subsequent recrystallization from alcoholic solvent such as ethanol provides 4-bromocinnoline.

Compounds of formula 2 are either commercially available or can be synthesized by methods well known in the art. For example, compounds of formula² where R¹ and R² are same can be prepared by treating 3,4-dihydroxy-acetophenone with the desired R¹LG reagent where R¹ is as defined above and LG is a suitable leaving group in the presence of a base such as cesium carbonate, triethylamine, sodium hydride, potassium carbonate, potassium hydride, and the like to provide the dialkylated product. Suitable organic solvents include acetone, acetonitrile, DMF, THF, and the like. 2-Amino-4,5-disubstituted acetophenones 2 is then prepared by nitration of 4,5-disubstituted acetophenones obtained from above with nitric acid in one of several solvents including acetic acid or sulfuric acid at ice bath temperatures to provide the corresponding 2-nitro-4,5-disubstituted acetophenones (see Mitzuta et al., Bioorg. Med. Chem. 10:675-683, 2002). Reduction of the nitro group under known reaction conditions e.g., hydrogenation with palladium on carbon, iron powder in acetic acid, or nickel boride, among others, provides the desired compound 2 (see Castle et al., J. Org. Chem. 19:1117, 1954).

Compounds of formula 2 where R¹ and R² are different can be prepared by selectively protecting 3,4-dihydroxyacetophenone the 4-hydroxy group as its 4-benzyl ether (see Greenspan et al., J. Med. Chem. 42:164, 1999) by treatment with benzyl bromide and lithium carbonate in DMF solution. Functionalization of the 3-OH group with the desired R¹LG where R¹ and LG are as defined above can be accomplished under the alkylation conditions described above, including Mitsunobu reaction. Removal of the benzyl ether by hydrogenolysis with palladium on carbon in alcoholic solvents such as methanol and followed by alkylation of the 4-OH yields with the desired R²LG group would provide the desired 3,4-disubstitutedacetophenones, which upon nitration, followed by reduction of the nitro group provides the desired compound 2.

Compounds of formula 1 where X and Z are nitrogen, Y is carbon, and R¹, R², R³ and R⁴ are as defined in the Detailed Description of the Invention can be prepared as described below.

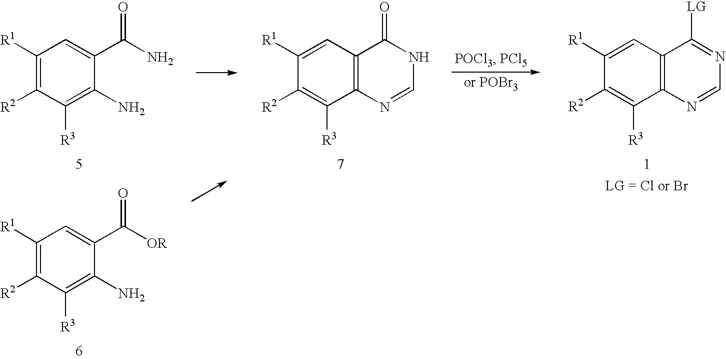

Reaction of 2-aminobenzamide compounds of formula 5 with trimethyl orthoformate or 2-aminobenzoic ester compounds of formula 6 with formamide in the presence of a base such as ammonium carbonate provides the corresponding 4-hydroxyquinazolone 7 which upon treatment with either phosphorous oxychloride or phosphorous oxybromide provides the corresponding chloro or bromo compound of formula 1. The chloro derivative may be prepared by heating 7 in neat phosphorous oxychloride, followed by recrystallization of the product after neutralization (see, for example, Castle et al., J. Org. Chem. 17:1571, 1952). The bromo derivative may be prepared by mixing a concentrated suspension of the 4-hydroxyquinazoline 7 in chloroform and phosphorous oxybromide at room temperature and then warming to reflux for 8 to 16 h. Extractive workup after neutralization and subsequent recrystallization from alcoholic solvent such as ethanol provides 4-bromoquinazoline 1. Compound 1 is then converted to a compound of Formula (I) as described in Scheme 1 above. Compounds of formula 5 and 6 are either commercially available or can be synthesized by methods common to the art.

Compounds of formula 1 where Y and Z are nitrogen, X is carbon, and R¹, R², and R³ are as defined in the Detailed Description of the Invention can be prepared as described as shown below.

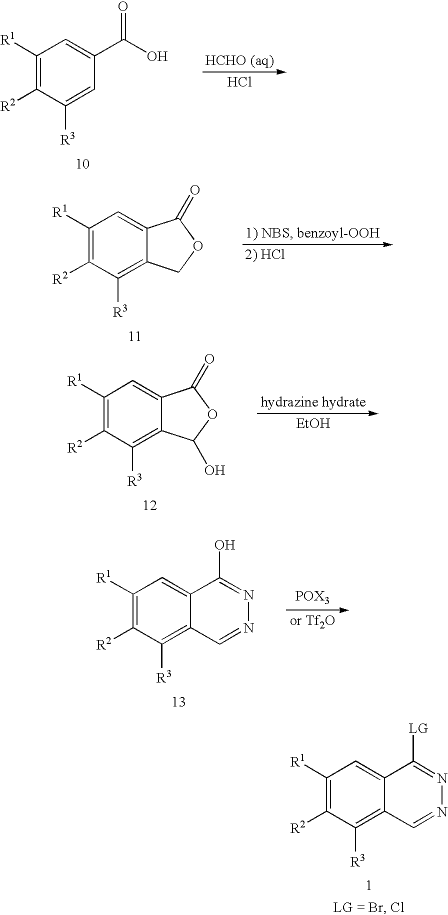

Treatment of a compound of formula 10 with aqueous formaldehyde and hydrochloric acid provides the cyclized ester 11. Compounds of formula 10 are either commercially available (e.g., 3,4-dimethoxy benzoic acid) or can be synthesized by methods common to the art (see, for example, Napoletano et al., Bioorg. Med. Chem. Lett. 11:33-37, 2001). Oxidation of 11 with a suitable oxidizing agent such as perbenzoic acid in the presence of N-bromosuccinimide, followed by treatment with hydrazine, provides 4-hydroxy phthalazines 13. Treatment of 13 with phosphorous oxyhalide or with triflic anhydride as described in Scheme 1 above provides the 4-halo phthalazines 1. Compound 1 is then converted to compound of Formula (I) where Y and Z are nitrogen and X is —CH═ as described in Scheme 1 above.

Compounds of Formula (I), where X is nitrogen, Y and Z are —CH═, and R¹, R², R³ and R^{3a} are as defined in the Detailed Description of the Invention, can be prepared as described in Scheme 3 below (see, J. Med. Chem., 42:5369, 1999).

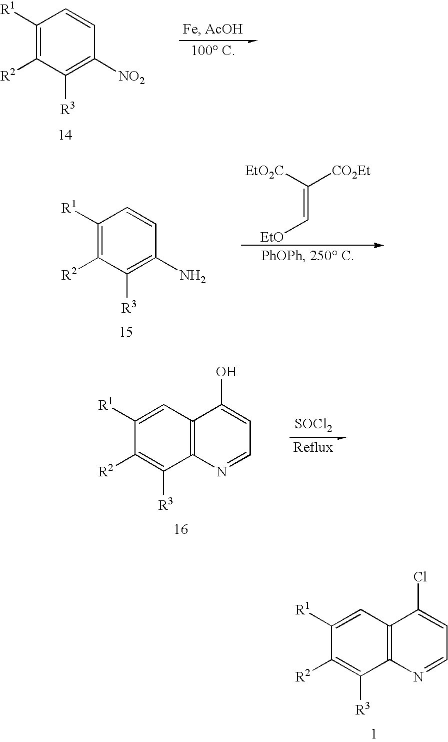

Compounds 14, where R³ is hydrogen, and R¹ and R² are the same and are selected from alkoxy, hydroxy, for example, methoxy, can be synthesized by methods common to the art. For example, 3,4-dihydroxy-nitrobenzene 14 (R³═H, R¹═R²═OH) can be treated with a desired R¹LG, where R¹ is as defined above and LG is a suitable leaving group, in the presence of a base, such as cesium carbonate, triethylamine, sodium hydride, potassium carbonate, potassium hydride, or the like, to provide the corresponding dialkoxy product. Suitable organic solvents include acetone, acetonitrile, DMF, THF, and the like. Reduction of the nitro group under known reaction conditions, e.g., hydrogenation with palladium on carbon, iron powder in acetic acid, or nickel boride, provides the amino compound 15 (see, Castle et al., J. Org. Chem. 19:1117, 1954).

Compounds 15, where R³ is hydrogen, R² is hydroxy, and R¹ is methoxy, can be prepared from 2-methoxy-5-nitrophenol as a starting material. Heating compound 15 with diethyl 2-(ethoxymethylene)malonate in the presence of diphenylether provides 4-hydroxyquinoline 16, which is then converted to 4-halo compound 1. Compound 1 is converted to a compound of Formula (I) as described in Scheme 1 above.

Compounds of formula 1, where Z is nitrogen; and X and Y are —CH═ and R¹, R², and R³ are as defined in the Detailed Description of the Invention, can be prepared as described below.

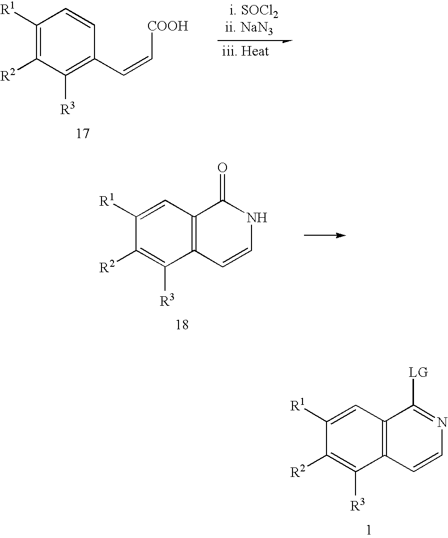

Treatment of acrylic acid derivative 17 with a halogenating agent, such as thionyl chloride, followed by treatment with sodium azide, provides acryloyl azide, which upon heating at approximately 270° C. in a suitable high boiling solvent, such as diphenylether, cyclizes to form the corresponding 2H-isoquinolin-1-one 18. Compounds of formula 17 are either commercially available or can be synthesized by methods common to the art. Compound 18 is then converted to compound 1, where LG is chloro or bromo, by treatment with phosphorus oxychloride or phosphorous oxybromide, respectively. Compound 1 is converted into a compound of Formula (I) as described in Scheme 1 above.

### Utility and Methods of Use

Provided herein are methods for treating a disorder or disease by inhibiting PDE10 enzyme. The methods, in general, comprises the step of administering a therapeutically effective amount of a compound of Formula (I), or an individual stereoisomer, a mixture of stereoisomers, or a pharmaceutically acceptable salt or solvate thereof, to a patient in need thereof to treat the disorder or disease.

In certain embodiments, this invention provides a use of a compound as described herein in the manufacture of a medicament for treating a disorder or disease treatable by inhibition of PDE10.

The compounds of the present invention inhibit PDE10 enzyme activity, and hence raise the levels of cAMP or cGMP within cells that express PDE10. Accordingly, inhibition of PDE10 enzyme activity would be useful in the treatment of diseases caused by deficient amounts of cAMP or cGMP in cells. PDE10 inhibitors would also be of benefit in cases wherein raising the amount of cAMP or cGMP above normal levels results in a therapeutic effect. Inhibitors of PDE10 may be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex, and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention are suitable for use in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways (Saxena et al., Br. J. Psychiatry Suppl, 35:26-37, 1998). Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention are therefore suitable for use in the indication of OCD. OCD may result, in some cases, from streptococcal infections that cause autoimmune reactions in the basal ganglia (Giedd et al., Am J Psychiatry. 157:281-283, 2000). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that, since PDE10 degrades cAMP or cGMP, the level of this enzyme affects memory in animals, for example, in humans. A compound that inhibits cAMP phosphodiesterase (PDE) can thereby increase intracellular levels of cAMP, which in turn activate a protein kinase that phosphorylates a transcription factor (cAMP response binding protein). The phosphorylated transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention are suitable for use in treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention are suitable for use in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 (also called Machado-Joseph disease or MJD) (ataxin-3); spinocerebellar ataxia type-6 (alpha 1a-voltage dependent calcium channel); spinocerebellar ataxia type-7 (ataxin-7); and spinal and bulbar muscular atrophy (SBMA, also know as Kennedy disease).

The basal ganglia are important for regulating the function of motor neurons; disorders of the basal ganglia result in movement disorders. Most prominent among the movement disorders related to basal ganglia function is Parkinson's disease (Obeso et al., Neurology. 62(1 Suppl 1):S17-30, 2004). Other movement disorders related to dysfunction of the basal ganglia include tardive dyskinesia, progressive supranuclear palsy and cerebral palsy, corticobasal degeneration, multiple system atrophy, Wilson disease, dystonia, tics, and chorea. The compounds of the invention are also suitable for use to treat movement disorders related to dysfunction of basal ganglia neurons.

PDE10 inhibitors are useful in raising cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis.

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity inhibits cell growth by raising cAMP. In some cases, PDE10 may be expressed in the transformed, cancerous cell but not in the parent cell line. In transformed renal carcinoma cells, PDE10 is expressed and PDE10 inhibitors reduce the growth rate of the cells in culture. Similarly, breast cancer cells are inhibited by administration of PDE 10 inhibitors. Many other types of cancer cells may also be sensitive to growth arrest by inhibition of PDE10. Therefore, compounds disclosed in this invention can be used to stop the growth of cancer cells that express PDE10.

The compounds of the invention are also suitable for use in the treatment of diabetes and related disorders such as obesity, by focusing on regulation of the cAMP signaling system. By inhibiting PDE-10, especially PDE-1 OA, intracellular levels of cAMP are increased, thereby increasing the release of insulin-containing secretory granules and, therefore, increasing insulin secretion. See, for example, WO 2005/012485, which is hereby incorporated by reference in its entirety. The compounds of Formula (I) can also be used to treat diseases disclosed in US Patent application publication No. 2006/019975, the disclosure of which is incorporated herein by reference in its entirety.

### Testing

The PDE10 inhibitory activities of the compounds of the present invention can be tested, for example, using the in vitro and in vivo assays described in the Biological Examples below.

### Administration and Pharmaceutical Compositions

In general, the compounds of this invention can be administered in a therapeutically effective amount by any of the accepted modes of administration for agents that serve similar utilities. The actual amount of a compound of this invention, i.e., the active ingredient, depends upon numerous factors, such as the severity of the disease to be treated, the age and relative health of the subject, the potency of the compound used, the route and form of administration, and other factors.

Therapeutically effective amounts of compounds of formula (I) may range from approximately 0.1-1000 mg per day; preferably 0.5 to 250 mg/day, more preferably 3.5 mg to 70 mg per day.

In general, compounds of this invention can be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors, such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, Gennaro, A. R. (Mack Publishing Company, 18th ed., 1995).

The level of the compound in a formulation can vary within the full range employed by those skilled in the art. Typically, the formulation contains, on a weight percent (wt %) basis, from about 0.01-99.99 wt % of a compound of Formula (I) based on the total formulation, with the balance being one or more suitable pharmaceutical excipients. Preferably, the compound is present at a level of about 1-80 wt %.

The compounds can be administered as the sole active agent or in combination with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range, and can be administered either simultaneously or sequentially.

Drugs suitable in combination with the compounds of the present invention include, but are not limited to, other suitable schizophrenia drugs such as Clozaril, Zyprexa, Risperidone, and Seroquel; bipolar disorder drugs, including, but not limited to, Lithium, Zyprexa, and Depakote; Parkinson's disease drugs, including, but not limited to, Levodopa, Parlodel, Permax, Mirapex, Tasmar, Contan, Kemadin, Artane, and Cogentin; agents used in the treatment of Alzheimer's disease, including, but not limited to, Reminyl, Cognex, Aricept, Exelon, Akatinol, Neotropin, Eldepryl, Estrogen and Cliquinol; agents used in the treatment of dementia, including, but not limited to, Thioridazine, Haloperidol, Risperidone, Cognex, Aricept, and Exelon; agents used in the treatment of epilepsy, including, but not limited to, Dilantin, Luminol, Tegretol, Depakote, Depakene, Zarontin, Neurontin, Barbita, Solfeton, and Felbatol; agents used in the treatment of multiple sclerosis, including, but not limited to, Detrol, Ditropan XL, OxyContin, Betaseron, Avonex, Azothioprine, Methotrexate, and Copaxone; agents used in the treatment of Huntington's disease, including, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g. agonists, antagonists, such as Rosiglitazone, Troglitazone and Pioglitazone), insulin secretagogues (e.g., sulfonylurea drugs, such as Glyburide, Glimepiride, Chlorpropamide, Tolbutamide, and Glipizide, and non-sulfonyl secretagogues), α-glucosidase inhibitors (such as Acarbose, Miglitol, and Voglibose), insulin sensitizers (such as the PPAR-γ agonists, e.g., the glitazones, biguanides, PTP-1B inhibitors, DPP-IV inhibitors, and 11beta-HSD inhibitors), hepatic glucose output lowering compounds (such as glucagon antagonists and metaformin, e.g., Glucophage and Glucophage XR), insulin and insulin derivatives (both long and short acting forms and formulations of insulin); and anti-obesity drugs, including, but not limited to, β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat).

### EXAMPLES

The following preparations and examples are given to enable those skilled in the art to more clearly understand and to practice the present invention. They should not be considered as limiting the scope of the invention, but merely as being illustrative and representative thereof.

All NMR spectra were recorded at 300 MHz on a Bruker Instruments NMR unless otherwise stated. Coupling constants (J) are in Hertz (Hz) and peaks are listed relative to TMS (δ0.00 ppm). Microwave reactions were performed using a Personal Chemistry Optimizer microwave reactor in Personal Chemistry microwave reactor vials. Sulfonic acid ion exchange resins (SCX) were purchased from Varian Technologies. Analytical HPLC was performed on 4.6 mm×100 mm Waters Sunfire RP C18 5 μm column. 4-Bromo-6,7-dimethoxyquinoline, a starting material for making certain compounds of Formula (I), is commercially available.

### Synthetic Examples

### Example 1

### Synthesis of 2-(1-(5-(6,7-dimethoxycinnolin-4-yl)-3-methylpyridin-2-yl)piperidin-4-yl)propan-2-ol

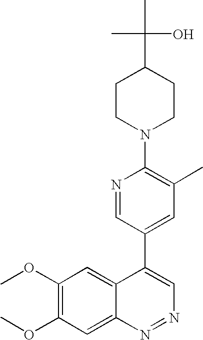

Step 1. To 100 mL round-bottomed flask was added 4-bromo-6,7-dimethoxycinnoline (15.4 g, 57 mmol), 6-fluoro-5-methylpyridin-3-ylboronic acid (10.1 g, 71 mmol), and trans-dichlorobis(triphenyl-phosphine)palladium (II) (3.4 g, 4.6 mmol) in 1,2-dimethoxyethane. An aqueous solution of sodium carbonate (27 g, 257 mmol) was added and the temperature was brought to 80° C. stir overnight. Upon completion, the reaction mixture was diluted with water (10 mL) and extracted with ethyl acetate. The organic extract was washed with water, saturated sodium chloride solution, dried with magnesium sulfate, filtered, and concentrated. The crude product was adsorbed onto a plug of silica gel and chromatographed through a Biotage pre-packed silica gel column eluting with a gradient of 1% to 5% methanol in dichloromethane, to provide 4-(6-fluoro-5-methylpyridin-3-yl)-6,7-dimethoxycinnoline.

Step 2. To a 50 mL round-bottomed flask was added ethyl isonipecotate (5.0 g, 32 mmol) in dimethylformamide at 0° C. Sodium hydroxide (0.86 g, 35 mmol) was added and the reaction mixture was allowed to stir for 15 minutes. 1-(Bromomethyl)benzene (4.2 ml, 35 mmol) was then added and the reaction mixture was allowed to stir slowly warming to room temperature. Upon completion, the reaction mixture was diluted with water and extracted with ethyl acetate. The organic extract was washed with water, saturated sodium chloride solution, dried with magnesium sulfate, filtered, and concentrated. The ethyl 1-benzylpiperidine-4-carboxylate product (5.8 g, 23.3 mmol) was then added to a 500 mL round-bottomed flask in THF at −78° C. Methyllithium (18.7 ml, 46.7 mmol) was added and allowed to stir slowly warming to room temperature overnight. Upon completion, the reaction mixture was concentrated. The residue was diluted with water and extracted with ethyl acetate. The organic extract was washed with saturated sodium carbonate solution, saturated sodium chloride solution, dried with magnesium sulfate, filtered, and concentrated. The crude product was adsorbed onto a plug of silica gel and chromatographed through a Biotage pre-packed silica gel column (40M) X2, eluting with a gradient of 1% to 10% methanol in dichloromethane, to provide 2-(1-benzylpiperidin-4-yl)propan-2-ol. To a solution of 2-(1-benzylpiperidin-4-yl)propan-2-ol in methanol was added palladium, 10 wt. % on activated carbon. A hydrogen balloon was attached to the reaction vessel and the reaction mixture was stirred overnight. Upon completion, the reaction mixture was filtered through Celite. The filtrate was concentrated to produce 2-(piperidin-4-yl)propan-2-ol.

Step 3. In a 25 mL round bottom flask was placed 4-(6-fluoro-5-methylpyridin-3-yl)-6,7-dimethoxycinnoline (0.0924 g, 0.309 mmol) and 2-(piperidin-4-yl)propan-2-ol (0.4383 g, 3.09 mmol) in dimethylsulfoxide at 90° C. to stir overnight. Upon completion, the reaction mixture was diluted with water and extracted with ethyl acetate. The organic extract was washed with water, saturated sodium chloride, dried with magnesium sulfate, filtered, and concentrated. The crude product was adsorbed onto a plug of silica gel and chromatographed through a Biotage pre-packed silica gel column (40S), eluting with a gradient of 1% to 5% methanol in dichloromethane, to provide 2-(1-(5-(6,7-dimethoxycinnolin-4-yl)-3-methylpyridin-2-yl)piperidin-4-yl)propan-2-ol (0.0913 g, 70.0% yield, M+1=423.3).

### Example 2

### Synthesis of 2-(1-(5-(6,7-dimethoxyquinolin-4-yl)-3-methylpyridin-2-yl)piperidin-4-yl)-propan-2-ol

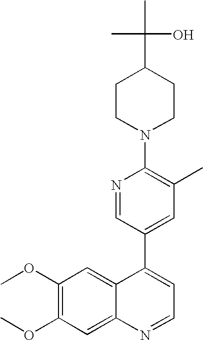

Step 1. To 100 mL round-bottomed flask was added 4-chloro-6,7-dimethoxyquinoline (0.1125 g, 0.503 mmol), 6-fluoro-5-methylpyridin-3-ylboronic acid (0.1075 g, 0.629 mmol), and trans-dichlorobis(triphenyl-phosphine)palladium (II) (0.0321 g, 0.0402 mmol) in 1,2-dimethoxyethane. An aqueous solution of sodium carbonate (27 g, 257 mmol) was added and the temperature was brought to 80° C. stir overnight. Upon completion, the reaction mixture was diluted with water and extracted with ethyl acetate. The organic extract was washed with water, saturated sodium chloride solution, dried with magnesium sulfate, filtered, and concentrated. The crude product was adsorbed onto a plug of silica gel and chromatographed through a Biotage pre-packed silica gel column (25M), eluting with a gradient of 10% to 70% ethyl acetate in hexane, to provide 4-(6-fluoro-5-methylpyridin-3-yl)-6,7-dimethoxyquinoline (0.059 g).

Step 2. In a 25 mL round bottom flask was placed 4-(6-fluoro-5-methylpyridin-3-yl)-6,7-dimethoxyquinoline (0.0454 g, 0.15 mmol) and 2-(piperidin-4-yl)propan-2-ol (0.1105 g, 0.76 mmol) to stir in DMSO at 90° C. overnight. Upon completion, the reaction mixture was diluted with water and extracted with ethyl acetate. The organic extract was washed with water, saturated sodium chloride solution, dried with magnesium sulfate, filtered, and concentrated. The crude product was adsorbed onto a plug of silica gel and chromatographed through a Biotage pre-packed silica gel column, eluting with a gradient of 1% to 5% methanol in dichloromethane, to provide 2-(1-(5-(6,7-dimethoxyquinolin-4-yl)-3-methylpyridin-2-yl)piperidin-4-yl)propan-2-ol (0.0478 g, 75% yield, m+1=422.3).

### Example 3

### Synthesis of 1-(5-(6,7-dimethoxycinnolin-4-yl)pyridin-2-yl)-4-(pyridin-2-yl)piperidin-4-ol

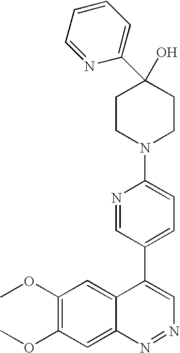

Step 1. To a 50 mL round-bottomed flask was added 4-bromo-6,7-dimethoxycinnoline (1.1 g, 3.718 mmol), 6-fluoropyridin-3-ylboronic acid (0.58 g, 4.090 mmol) and palladium tetrakis (0.21 g, 0.1859 mmol) in 1,2-dimethoxyethane. An aqueous solution of cesium carbonate (3.26 g, 10.04 mmol) in water (42 mL) was added and the temperature was brought to 80° C. for 2 hours. Upon completion, the reaction mixture was diluted with water and extracted with ethyl acetate. The organic extract was washed with water, saturated sodium chloride solution, dried with magnesium sulfate, filtered, and concentrated. The crude product was adsorbed onto a plug of silica gel and chromatographed through a Biotage pre-packed silica gel column (40M), eluting with a gradient of 1% to 5% methanol in dichloromethane, to provide 4-(6-fluoropyridin-3-yl)-6,7-dimethoxycinnoline.

Step 2. In a 25 mL round bottom flask was placed 4-(6-fluoropyridin-3-yl)-6,7-dimethoxycinnoline (0.16 g, 0.57 mmol), 4-(pyridin-2-yl)piperidin-4-ol dihydrochloride (0.50 g, 2.8 mmol), and potassium carbonate (0.36 ml, 6.0 mmol) in dimethylsulfoxide at 90° C. to stir overnight. Upon completion, the reaction mixture was diluted with water (10 mL) and extracted with ethyl acetate. The organic extract was washed with water, saturated sodium chloride solution, dried with magnesium sulfate, filtered, and concentrated. The crude product was adsorbed onto a plug of silica gel and chromatographed through a Biotage pre-packed silica gel column (25M), eluting with a gradient of 1% to 5% methanol in dichloromethane, to provide 1-(5-(6,7-dimethoxycinnolin-4-yl)pyridin-2-yl)-4-(pyridin-2-yl)piperidin-4-ol (0.1393 g, 55% yield, M+1=444.2).

### Example 4

### Synthesis of 1-(5-(6,7-dimethoxycinnolin-4-yl)-3-methylpyridin-2-yl)-4-(pyridin-2-yl)-piperidin-4-ol

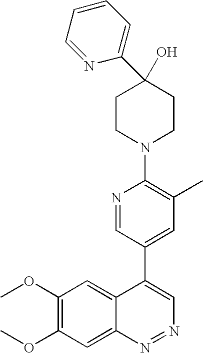

In a 25 mL round bottom flask was placed 4-(6-fluoro-5-methylpyridin-3-yl)-6,7-dimethoxycinnoline and 4-(pyridin-2-yl)piperidin-4-ol (0.52 g, 2.90 mmol) in dimethylsulfoxide at 90° C. to stir overnight. Upon completion, the reaction mixture was diluted with water and extracted with ethyl acetate. The organic extract was washed with water, saturated sodium chloride solution, dried with magnesium sulfate, filtered, and concentrated. The crude product was adsorbed onto a plug of silica gel and chromatographed through a Biotage pre-packed silica gel column (25 M), eluting with a gradient of 1% to 5% methanol in dichloromethane, to provide 1-(5-(6,7-dimethoxycinnolin-4-yl)-3-methylpyridin-2-yl)-4-(pyridin-2-yl)piperidin-4-ol (0.1502 g, M+1=458.1).

### Example 5

### Synthesis of 1-(3-chloro-5-(6,7-dimethoxycinnolin-4-yl)pyridin-2-yl)-4-(pyridin-2-yl)piperidin-4-ol

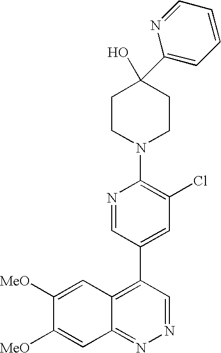

Step 1. 4-(Pyridin-2-yl)piperidin-4-ol (982 mg, 5.509 mmol) and 5-bromo-2,3-dichloro-pyridine (250 mg, 1.102 mmol) were combined in DMSO (5 mL) and the reaction mixture was heated to 110° C. overnight. The reaction mixture was cooled to room temperature, diluted with dichloromethane and water, the layers were separated and the aqueous was extracted with dichloromethane. The combined organics were washed with brine, dried over sodium sulfate, filtered and concentrated to give 1-(5-bromo-3-chloropyridin-2-yl)-4-(pyridin-2-yl)piperidin-4-ol as a viscous oil which was used without further purification.

Step 2. A mixture of 1-(5-bromo-3-chloropyridin-2-yl)-4-(pyridin-2-yl)piperidin-4-ol (406 mg, 1.10 mmol), 4,4,5,5-tetramethyl-2-(4,4,5,5-tetramethyl-1,3,2-dioxaborolan-2-yl)-1,3,2-dioxaborolane (336 mg, 1.32 mmol), potassium acetate (292 mg, 2974 μmol), and 1,1′-bis(diphenylphosphino)ferrocene-palladium dichloride (56.4 mg, 77.1 μmol) in dioxane was heated to 110° C. for several hours. LCMS analysis showed complete conversion to the boronic acid. The reaction mixture was cooled to room temperature, filtered through celite rinsing with dichloromethane, concentrated to give 5-chloro-6-(4-hydroxy-4-(pyridin-2-yl)piperidin-1-yl)pyridin-3-ylboronic acid which was used directly in the next step.

Step 3. To a solution of 4-bromo-6,7-dimethoxycinnoline (70 mg, 260 μmol), 5-chloro-6-(4-hydroxy-4-(pyridin-2-yl)piperidin-1-yl)pyridin-3-ylboronic acid (87 mg, 260 mmol), and trans-dichlorobis(triphenyl-phosphine)palladium (ii) (183 mg, 260 μmol) in dimethoxyethane (9.8 ml) was added an aqueous solution of cesium carbonate (85 mg, 260 mmol). The reaction mixture was heated to 80° C. for two hours after which time complete product formation was observed by LCMS. The reaction mixture was cooled to room temperature, diluted with water and ethyl acetate, the layers were separated and the aqueous was extracted with ethyl acetate. The combined organics were washed with brine, dried over sodium sulfate, filtered and concentrated. The residue was adsorbed onto a plug of silica gel and chromatographed through a Biotage pre-packed silica gel column (25M), eluting with a gradient of 2% to 9% methanol in dichloromethane to give the title compound (M+1=478.1).

### Example 6

### Synthesis of 4-cyclopropyl-1-(5-(6,7-dimethoxycinnolin-4-yl)-3-methylpyridin-2-yl)piperidin-4-ol

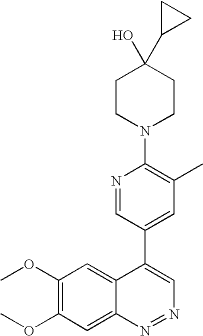

Step 1. A solution of 1,4-dioxa-8-azaspiro[4.5]decane (3 ml, 18 mmol), 4-(6-fluoropyridin-3-yl)-6,7-dimethoxycinnoline (0.5 g, 2 mmol), in DMSO (0.2M) was heated to 100° C. overnight. LC/MS of the reaction mixture the next day showed formation of new peak that contained the desired product mass. The residue was diluted with dichloromethane and washed with water and brine. The organic layers were combined and loaded directly onto a Biotage samplet for silica gel purification. The Biotage column was eluted with (0-5% MeOH/DCM) to give 4-[6-(1,4-dioxa-8-azaspiro[4.5]dec-8-yl)-5-methylpyridine-3-yl]-6,7-dimethoxycinnoline (0.44 g) as a yellow solid.

Step 2. A solution of 4-[6-(1,4-dioxa-8-azaspiro[4.5]dec-8-yl)-5-methylpyridine-3-yl]-6,7-dimethoxycinnoline in (1:1) TFA and DCM was heated at 50° for 2 days until LC/MS showed most of the starting material had disappeared. The reaction mixture was rotovapped to remove most of the TFA. The residue was diluted with DCM and then quenched by stirring vigorously with saturated sodium bicarbonate till the mixture had neutralized. The layers were separated with separatory funnel. The organic layer was washed two more times with saturated bicarbonate solution. The organic layer was dried over sodium sulfate and rotovapped to afford 1-(5-(6,7-dimethoxycinnolin-4-yl)-3-methylpyridin-2-yl)piperidin-4-one as brown oil.

Step 3. To 1-(5-(6,7-dimethoxycinnolin-4-yl)-3-methylpyridin-2-yl)piperidin-4-one (1.1 g, 3 mmol) was added cerium chloride (0.7 g, 3 mmol) and anhydrous THF. The resulting suspension was sonicated for at least 15 minutes. The reaction mixture was cooled in a 0° C. bath for 30 min before dropwise addition of cyclopropylmagnesium bromide (8 ml, 4 mmol). The resulting mixture was stirred at 0° C. for another 30 min then allowed to warm to room temperature while stirring overnight. LC/MS showed formation of desired product peak. Reaction mixture was quenched with methanol and then loaded onto Biotage samplets for silica gel chromatography. Purification by Biotage (0-10% MeOH/DCM) produced rich cut that was about 89% pure. A second purification by Biotage (4% MeOH/DCM) produced title compound (M+1=421.2).

### Example 7

### Synthesis of 1-(5-(6,7-dimethoxycinnolin-4-yl)-3-methylpyridin-2-yl)-4-methylpiperidin-4-ol

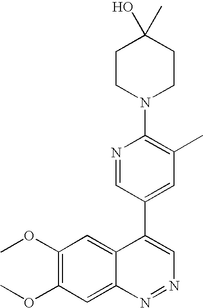

Step 1. A solution of 4-[6-(1,4-dioxa-8-azaspiro[4.5]dec-8-yl)-5-methylpyridine-3-yl]-6,7-dimethoxycinnoline in (1:1) TFA and DCM was heated at 50° for 2 days until LC/MS showed most of the starting material had disappeared. The reaction mixture was rotovapped to remove most of the TFA. The residue was diluted with DCM and then quenched by stirring vigorously with saturated sodium bicarbonate till the mixture was neutralized. The layers were separated with separatory funnel. The organic layer was washed two more times with saturated bicarbonate solution. The organic layer was dried over sodium sulfate and rotovapped to afford 1-(5-(6,7-dimethoxycinnolin-4-yl)-3-methylpyridin-2-yl)piperidin-4-one as brown oil.

Step 2. To a suspension of 1-(5-(6,7-dimethoxycinnolin-4-yl)-3-methylpyridin-2-yl)piperidin-4-one (0.6 g, 2 mmol) in anhydrous THF was added dropwise methylmagnesium bromide (1 ml, 2 mmol) at 0° C. Another 10 mL of THF was added to the reaction mixture. The ice bath was removed upon completion of addition and the reaction mixture was allowed to stir at room temperature overnight. The reaction was quenched by addition of methanol and directly loaded onto a Biotage samplet for silica gel chromatography. Purification by Biotage (0-10% MeOH/DCM) produced a rich cut that still contained some impurity. The rich cut was purified a second time by prep-plate TLC (10% MeOH/DCM) to isolate the title product (M+1=395.2).

### Example 8

### Synthesis of 2-(1-(3-(difluoromethyl)-5-(6,7-dimethoxycinnolin-4-yl)pyridin-2-yl)piperidin-4-yl)propan-2-ol

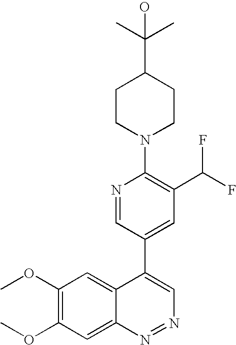

Step 1. To a solution of 5-chloro-2-fluoronicotinaldehyde (1 g, 6 mmol) in DCM (0.3M) was added Deoxo-Fluor 50% solution in THF (2 mL, 11 mmol) and ethyl alcohol (0.1 mL, 2 mmol) and the resulting mixture was stirred at room temperature overnight. The reaction mixture was directly loading onto a Biotage samplet for silica gel chromatography. Purification by Biotage (DCM) afforded 5-chloro-3-(difluoromethyl)-2-fluoropyridine as yellow oil.

Step 2. A mixture of 5-chloro-3-(difluoromethyl)-2-fluoropyridine (0.7 g, 4 mmol) and 2-(piperidin-4-yl)propan-2-ol (3 g, 19 mmol) in DMSO (6 mL) was heated to 90° C. overnight. The reaction mixture was diluted with DCM and washed with water and brine. The combined organic layers were directly loaded onto a Biotage samplet for silica gel chromatography. Purification by Biotage (0-10% MeOH/DCM) afforded 2-(1-(5-chloro-3-(difluoromethyl)pyridin-2-yl)piperidin-4-yl)propan-2-ol.

Step 3. A mixture of 2-(1-(5-chloro-3-(difluoromethyl)pyridin-2-yl)piperidin-4-yl)propan-2-ol (0.6 g, 1.99 mmol), bis(pinacolato)diboron (1.1 g, 4.39 mmol), potassium acetate (0.58 g, 5.98 mmol), tris(dibenzylideneacetone)dipalladium (0.04 g, 0.04 mmol), and S-phos (0.07 g, 0.160 mmol) in dioxane under nitrogen was heated to 110° C. for 4 hr. The reaction mixture was purified by Biotage silica gel chromatography (0-10% MeOH/DCM) to isolate 5-(difluoromethyl)-6-(4-(2-hydroxypropan-2-yl)piperidin-1-yl)pyridin-3-ylboronic acid.

Step 4. To a mixture of 5-(difluoromethyl)-6-(4-(2-hydroxypropan-2-yl)piperidin-1-yl)-pyridin-3-ylboronic acid (0.6 g, 2 mmol), 4-bromo-6,7-dimethoxycinnoline (0.5 g, 2 mmol), and palladium tetrakistriphenylphosine (0.1 g, 0.1 mmol) was added dioxane (33 mL) and a premixed solution of cesium carbonate in water (15 mL, 0.3 M). The reaction flask was sealed and heated to 80° C. for 7 hr till LC/MS showed complete conversion. The reaction mixture was transferred into a separatory funnel and allowed to separate into two layers. The aqueous layer was removed. The organic layer was diluted with excess amount of DCM and washed multiple times with brine. The organic layer was directly loaded onto a Biotage samplet for silica gel chromatography. Purification by Biotage (0-10% MeOH/DCM) produced a rich cut that still contained some impurity by LC/MS. The rich cut was purified a second time by Biotage with a more gradual gradient (20-50% MeOH/DCM) produced the title compound (M+1=459.2).

### Example 9

### 4-(6-(4-(2-Hydroxypropan-2-yl)piperidin-1-yl)-5-methylpyridin-3-yl)-7-methoxycinnolin-6-ol

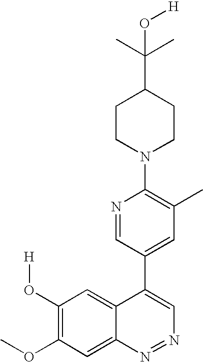

Step 1. To a solution of 4-bromo-6-fluoro-7-methoxycinnoline (1 g, 4 mmol) and (4-methoxyphenyl)methanol (0.5 g, 4 mmol) in DMF was added LHMDS (6 ml, 6 mmol) at room temperature. The mixture was stirred at room temperature overnight. The reaction was quenched with water, diluted with EtOAc, washed with water and brine. The organic layer was dried with sodium sulfate and concentrated by rotovap. Purification by Biotage™ (1-2.5% MeOH/DCM) isolated 6-(benzyloxy)-4-(6-fluoro-5-methylpyridin-3-yl)-7-methoxycinnoline.

Step 2. To a solution of 6-(4-methoxybenzyloxy)-4-bromo-7-methoxycinnoline (0.58 g, 1.5 mmol), 6-fluoro-5-methylpyridin-3-ylboronic acid (0.24 g, 1.5 mmol), sodium carbonate (3.1 mmol, 2M solution in water), and dimethoxyethane (5 mL) under an atmosphere of nitrogen was added trans-dichlorobis(triphenyl-phosphine)palladium (0.11 g, 0.15 mmol). The resulting mixture was heated to 80° C. for 5 hr. The mixture was cooled to room temperature and azeotroped with acetonitrile to remove water. The remaining residue was purified by Biotage™ (elution 0-10% MeOH/DCM) to isolate 6-(benzyloxy)-4-(6-fluoro-5-methylpyridin-3-yl)-7-methoxycinnoline.

Step 3. A solution of 6-(4-methoxybenzyloxy)-4-(6-fluoro-5-methylpyridin-3-yl)-7-methoxycinnoline (0.61 g, 1.5 mmol) in 6 mL DMSO was added 2-(piperidin-4-yl)propan-2-ol (1.1 g, 7.5 mmol) was heated to 90° C. overnight. The reaction mixture was cooled to room temperature, diluted with dichloromethane and washed with multiple water and brine washes to remove DMSO. The organic layer was dried over sodium sulfate and concentrated by rotovap evaporation. Purification by Biotage™ (eluted with 0-10% MeOH/DCM) to isolate 2-(1-(5-(6-(benzyloxy)-7-methoxycinnolin-4-yl)-3-methylpyridin-2-yl)piperidin-4-yl)propan-2-ol.

Step 4. To a solution of 2-(1-(5-(6-(4-methoxybenzyloxy)-7-methoxycinnolin-4-yl)-3-methylpyridin-2-yl)piperidin-4-yl)propan-2-ol (0.167 g, 0.32 mmol) in DCM was added 1 mL of water and DDQ. The resulting mixture was vigorously stirred at room temperature for 40 minutes. Reaction was quenched and basified with saturated bicarbonate solution. The mixture was extract with DCM, then concentrated. Purification by prep plate TLC (10% MeOH/DCM) isolated 4-(6-(4-(2-hydroxypropan-2-yl)piperidin-1-yl)-5-methylpyridin-3-yl)-7-methoxycinnolin-6-ol. (M=408)

### Example 10

### 4-(6-(4-(2-Fluoropropan-2-yl)piperidin-1-yl)-5-methylpyridin-3-yl)-6,7-dimethoxycinnoline

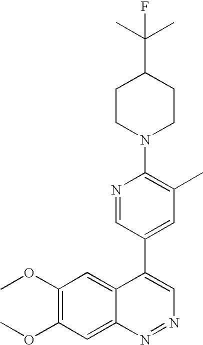

A solution of 2-(1-(5-(6,7-dimethoxycinnolin-4-yl)-3-methylpyridin-2-yl)piperidin-4-yl)propan-2-ol (140 mg, 331 μmol) in dichloromethane (1.2 ml) was cooled to −78° C. and a solution of diethylaminosulfur trifluoride (88 μl, 663 μmol) in dichloromethane (0.22 ml) was added dropwise. The reaction was stirred at −78° C. for 45 min, then warmed to 0° C. and stirred for 15 minutes after which time analysis by LCMS showed formation of desired product along with the tri-substituted olefin elimination product. The reaction mixture was allowed to warm to room temperature, diluted with dichloromethane and the layers were separated. The aqueous layer was extracted with dichloromethane (3×), washed with saturated sodium chloride, and dried over sodium sulfate. The solid obtained after work-up was triturated with MeOH (3×) to remove olefin by-product. The remaining mixture was subjected to flash silica gel chromatography using a 3.2-4.3% methanol/dichloromethane gradient over 10 column volumes to give a mixture. The material was recombined and purified by reverse-phase preparative HPLC using a Phenomenex Gemini™ column, 5 micron, 150×30 mm, 0.1% TFA in CH₃CN/H₂O, gradient 5% to 60% over 20 min to give 4-(6-(4-(2-fluoropropan-2-yl)piperidin-1-yl)-5-methylpyridin-3-yl)-6,7-dimethoxycinnoline and 6,7-dimethoxy-4-(5-methyl-6-(4-(propan-2-ylidene)piperidin-1-yl)pyridin-3-yl)cinnoline. (M+1=425.10)

| Structure | As in procedure: | Difference: | Difference: | MS + 1 |
|---|---|---|---|---|
| 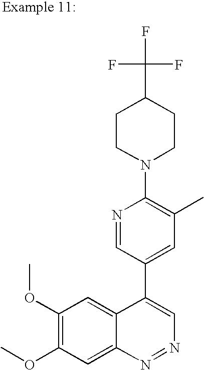 | Example 1 | 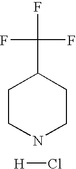 | | 433.1 |
| 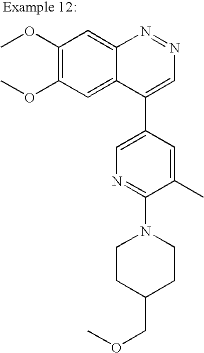 | Example 1 | 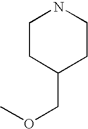 | | 408 |
| 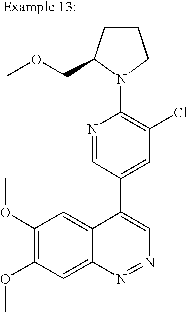 | Example 5 | 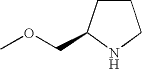 | | 415.1 |
| 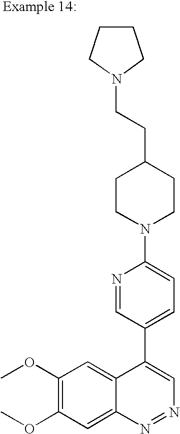 | Example 3(use 5 equivamine insteadof 10) | 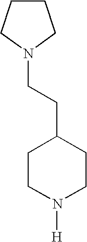 | | 448.0 |
| 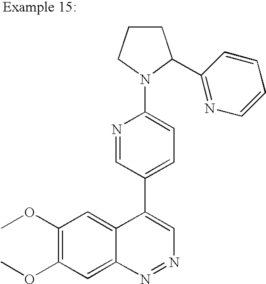 | Example 3(use 5 equivamine insteadof 10) | 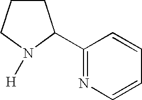 | | 414.0 |
| 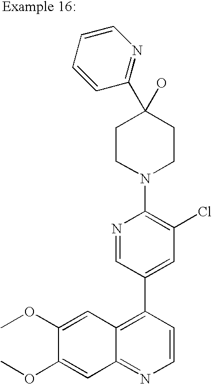 | Example 5 | 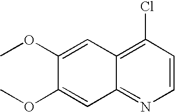 | | 477.2 |
| 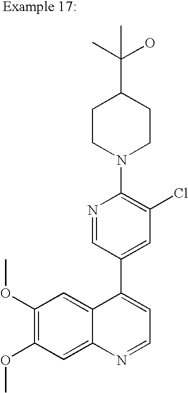 | Example 5,change 4-bromo-6,7-dimethoxycinnolinefor 4-chloro-6,7-dimethoxy-quinoline | 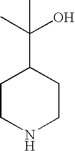 | 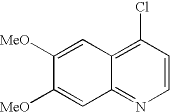 | 442 |
| 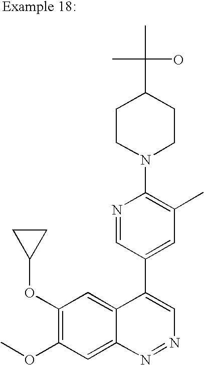 | Example 1 | 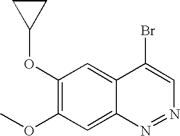 | | 449.2 |
| 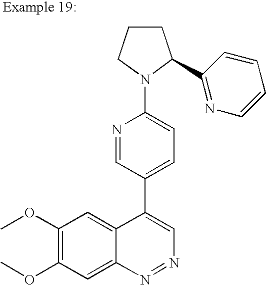 | Enantiomerseparated fromracemicExample 15 | Example 15 | | 414.0 |
| 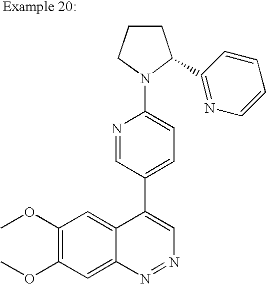 | Enantiomerseparated fromracemicExample 15 | Example 15 | | 414.0 |
| 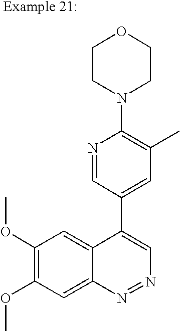 | Example 1 | 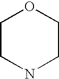 | | 367.2 |
| 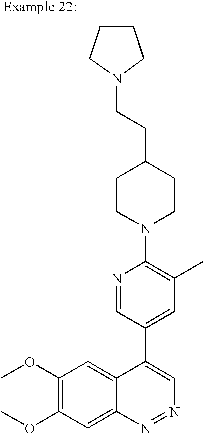 | Example 1(use 5 equivamine insteadof 10) | 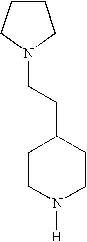 | | 462.4 |
| 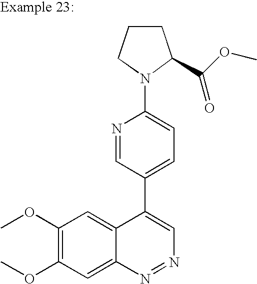 | Example 1(use 5 equivamine insteadof 10) | 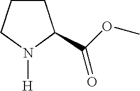 | | 395.2 |
| 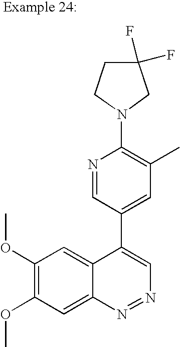 | Example 1 | 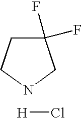 | | 387.2 |
| 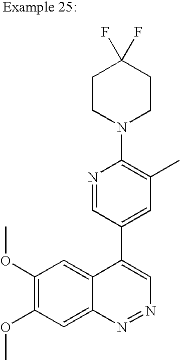 | Example 1 | 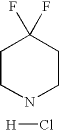 | | 401.2 |
| 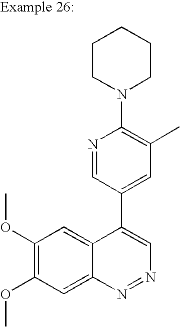 | Example 1 | 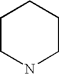 | | 365.2 |
| 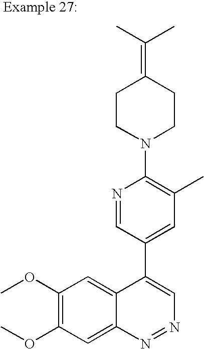 | Example 1 | 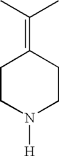 | | 405.1 |
| 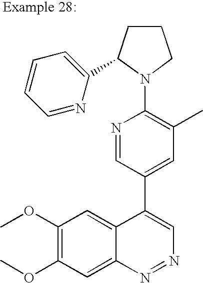 | Example 1(use 5 equivamine insteadof 10) | 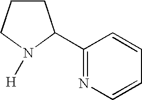 | | 428.0 |
| 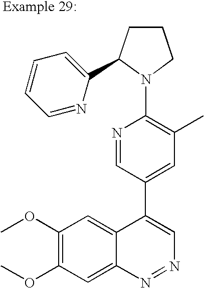 | Example 1(use 5 equivamine insteadof 10) | 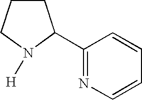 | | 428.0 |

### Biological Examples

### Example 1

### mPDE10A7 Enzyme Activity and Inhibition

Enzyme Activity. To analyze the enzyme activity, 5 μL of serial diluted mPDE10A7 containing lysate were incubated with equal volumes of diluted (100-fold) fluorescein labeled cAMP or cGMP for 30 min in MDC HE 96-well assay plates (Molecular Devices Corp., Sunnyvale Calif.) at room temperature. Both the enzyme and the substrates were diluted in the following assay buffer: Tris/HCl (pH 8.0) 50 mM, MgCl₂ 5 mM, 2-mercaptoethanol 4 mM, and BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP assay kit (Molecular Devices) was used to assess enzyme properties of mPDE10A7. Data were analyzed with SOFTMAX PRO software (Molecular Devices).

Enzyme Inhibition. To check the inhibition profile, 10 μL of serial diluted compounds were incubated with 30 μl of diluted PDE enzymes in a 96-well polystyrene assay plate for 30 min at room temperature. After incubation, 5 μL of the compound-enzyme mixture were aliquoted into a MDC HE black plate, mixed with 5 μl of 100-fold diluted fluorescein labeled substrates (cAMP or cGMP), and incubated for 30 min at room temperature. The reaction was stopped by adding 20 μL of diluted binding reagents and counted in an Analyst GT for fluorescence polarization. The data were analyzed with SoftMax Pro.

The IC₅₀ values of a representative number of compounds of the invention in the above assay are as follows:

| | IC₅₀ |
|---|---|
| Cpd # | (nm) |
| 2 | 5.58 |
| 4 | 2.8 |
| 7 | 0.6 |
| 8 | 3.07 |
| 9 | 1 |
| 10 | 1.87 |
| 11 | 2.2 |
| 12 | 14.71 |
| 13 | 1.02 |
| 14 | 416.83 |
| 15 | 126.14 |
| 16 | 11 |
| 17 | 10.19 |
| 18 | 6.56 |
| 20 | 60.25 |
| 22 | 4.85 |
| 23 | 24.89 |
| 24 | 4.22 |
| 25 | 7.42 |
| 27 | 5.63 |
| 28 | 2.08 |
| 30 | 9.57 |
| 32 | 139.84 |
| 33 | 60.59 |
| 35 | 191.11 |

### Example 2

### Apomorphine Induced Deficits in Prepulse Inhibition of the Startle Response in Rats, an in Vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g., the reflex activity of the rats is measured. The reflex of the rats to the burst of noise may be attenuated by preceding the startle stimulus with a stimulus of lower intensity, at 3 to 12 db above background (65 db), which attenuates the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine reduces the inhibition of the startle reflex produced by the prepulse. Antipsychotic drugs such as haloperidol prevents apomorphine from reducing the prepulse inhibition of the startle reflex. This assay can be used to test the antipsychotic efficacy of PDE10 inhibitors, as they reduce the apomorphine-induced deficit in the prepulse inhibition of startle.

### Example 3

### Conditioned Avoidance Responding (CAR) in Rats, an in Vivo Test for Antipsychotic Activity

Conditioned avoidance responding (CAR) occurs, for instance, when an animal learns that a tone and light predict the onset of a mild foot shock. The subject learns that when the when the light and tone are on it must leave the chamber and enter a safe area. All known antipsychotic drugs reduce this avoidance response at doses which do not cause sedation. Examining the ability of test compounds to suppress the conditioned avoidance has been widely used for close to fifty years to screen for drugs with useful antipsychotic properties.

In this example, an animal is placed in a two-chambered shuttle box and presented with a neutral conditioned stimulus (CS) consisting of a light and tone, followed by an aversive unconditioned stimulus (US) consisting of a mild foot shock through a floor grid in the shuttle box chamber. The animal is free to escape the US by running from one chamber to the other, where the grid is not electrified. After several presentations of the CS-US pair, the animal typically learns to leave the chamber during the presentation of the CS and avoid the US altogether. Animals treated with clinically-relevant doses of antipsychotic drugs have a suppression of their rate of avoidances in the presence of the CS even though their escape response to the shock itself is unaffected.

Specifically, conditioned avoidance training is conducted using a shuttle box (Med Associates, St. Albans, Vt.). The shuttle box is divided into 2 equal compartments that each contain a light source, a speaker that emits an 85 dB tone when activated and an electrified grid that can deliver a scrambled foot shock. Sessions consist of 20 trials per day (intertrial interval of 25-40 sec) during which a 10 sec illumination and a concurrent 10 sec tone signals the subsequent delivery of a 0.5 mA shock applied for a maximum of 10 sec. Active avoidance, defined as the crossing into the opposite compartment during the 10 sec conditioning stimuli (light and tone) prevents the delivery of the shock. Crossing over to the other compartment after the delivery of the shock terminates shock delivery and is recorded as an escape response. If an animal does not leave the conditioning chamber during the delivery of the shock it is recorded as an escape failure. Training is continued daily until the avoidance of 16 or more shocks out of 20 trials (80% avoidance) on 2 consecutive days is achieved. After this criterion is reached the rats are given one day of pharmacological testing. On test day, rats are randomly assigned to experimental groups, weighed and injected intraperitoneally i.p. (1 cc tuberculin syringe, 26⅜ gauge needle) or p.o. (18 gauge feeding needle) with either control or compound solutions. Compounds are injected at 1.0 ml/kg for i.p. and 10 ml/kg for p.o. administration. Compounds can be administered either acutely or chronically. For testing, each rat is placed in the shuttle box, and given 20 trials with the same parameters as described above for training trials. The number of avoidances, escapes, and escape failures are recorded.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I): 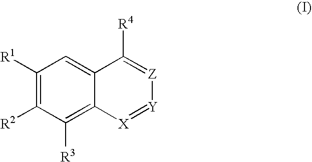
or an individual stereoisomer, a mixture of stereoisomers, or a pharmaceutically acceptable salt thereof, wherein:
one or two of X, Y and Z are —CH— and the remaining is —N—;
R¹ and R² are each independently selected from alkyl, hydroxy, or alkoxy;
R³ is hydrogen, alkyl, halo, or alkoxy;
R⁴ is a selected from formula (a) or (b): 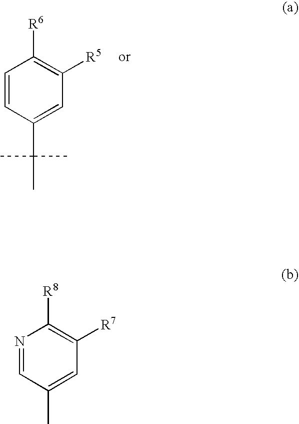
where:
R⁵ and R⁷ are independently hydrogen, alkyl, halo, or fluoroalkyl;
R⁶ and R⁸ are independently 5-8 membered monocyclic, saturated heterocyclyl substituted with one to three substituents independently selected from R^{a}, R^{b}, and R^{c} which are independently hydrogen, C₁₋₉alk, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, heterocyclylalkyl and optionally substituted heterocyclyl provided that: (i) when X and Y are N and Z is —CH═ or X and Y are —CH— and Z is —N—, then at least one of R^{a}, R^{b}, and R^{c} is not hydrogen; (ii) when X and Y are N and Z is —CH═, R⁴ is a group of formula (b) where R⁷ is hydrogen, then R⁸ is not 2-methylmorpholin-4-yl, 2,6-dimethylmorpholin-4-yl, 4-methylpiperazin-1-yl, 2,6-dimethylpiperazin-4-yl, 4-methoxypiperidin-1-yl, 4-fluoropiperidin-1-yl, 4,4-difluoropiperidin-1-yl, 4-methylaminopiperidin-1-yl, 4-hydroxy-4-phenylpiperidin-1-yl, 4-cyano-4-phenylpiperidin-1-yl, 4-hydroxypiperidin-1-yl, 1-tert-butoxycarbonylpyrrolidin-3-yl, or 3-methoxypyrrolidin-1-yl; and (iii) when X and Y are —CH— and Z is —N—, R⁴ is a group of formula (b) where R⁷ is hydrogen, then R⁸ is not 4-methoxypiperidin-1-yl or 2,6-dimethylmorpholin-4-yl.

2. The compound of claim 1 where R¹ and R² are alkoxy and R³ is hydrogen.

3. The compound of claim 1 wherein X is nitrogen, and Y and Z are ═CH—.

4. The compound of claim 2 where R¹ and R² are alkoxy and R³ is hydrogen.

5. The compound of claim 1 wherein X and Y are nitrogen and Z is —CH═.

6. The compound of claim 3 where R¹ and R² are alkoxy and R³ is hydrogen.

7. The compound of claim 1 where R⁴ is a group of formula (b).

8. The compound of claim 7 where R⁷ is hydrogen, halo, alkyl, or fluoroalkyl and R⁸ is piperidin-1-yl substituted as above.

9. The compound of claim 1 where R⁴ is a group of formula: 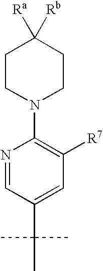
wherein R⁷ is hydrogen, halo, alkyl, or fluoroalkyl and R⁸ is piperidin-1-yl substituted with R^{a} and R^{b} where R^{a} is hydrogen, hydroxyl, halo, or alkoxy and R^{b} is alkyl, hydroxyalkyl, alkoxyalkyl, cycloalkyl, optionally substituted phenyl or optionally substituted heteroaryl.

10. The compound of claim 9 wherein R⁷ is hydrogen, chloro, methyl, or difluoromethyl and R⁸ is piperidin-1-yl substituted with R^{a} and R^{b} where R^{a} is hydrogen or hydroxyl and R^{b} is hydroxyalkyl, alkoxyalkyl, cycloalkyl, alkyl, or optionally substituted heteroaryl.

11. The compound of claim 9 wherein R^{a} is hydrogen or hydroxyl and R^{b} is —C(CH₃)(OH)CH₃, methyl, ethyl, cyclopropyl, cyclobutyl, or optionally substituted pyridin-2-yl.

12. The compound of claim 9 wherein R^{a} is hydrogen or hydroxyl and R^{b} is —C(CH₃)(OH)CH₃, methyl, cyclopropyl, or pyridin-2-yl.

13. A compound disclosed in Table 1 in the specification.

14. A pharmaceutical composition comprising a compound of claim 1 and a pharmaceutically acceptable excipient.

15. A method of treating a disorder treatable by inhibition of PDE10 in a patient which method comprises administering to the patient a pharmaceutical composition comprising a therapeutically effective amount of a compound of Formula (I), a pharmaceutically acceptable salt thereof, or a mixture of a compound of Formula (I) and a pharmaceutically acceptable salt thereof; and a pharmaceutically acceptable excipient.
